(19) United States
(12) Patent Application Publication
(10) Pub. No.: US 2022/0367007 A1
(43) Pub. Date: Nov. 17, 2022

(54) METHOD FOR GENERATING FUNCTIONAL PROTEIN SEQUENCES WITH GENERATIVE ADVERSARIAL NETWORKS

(71) Applicants: UAB BIOMATTER DESIGNS, Vilnius (LT); Vilnius University, Vilnius (LT)

(72) Inventors: Laurynas Karpus, Viln (LT); Vykintas Jauniskis, Vilnius (LT); Donatas Repecka, Vilnius (LT); Rolandas Meskys, Vilnius (LT)

(21) Appl. No.: 17/642,836

(22) PCT Filed: Sep. 10, 2019
(86) PCT No.: PCT/IB2020/058401

§ 371 Date: Mar. 14, 2022

(30) Foreign Application Priority Data
Sep. 27, 2019 (LT) LT2019 524

(51) Int. Cl. G16B 30/20; G16B 5/20; G16B 40/20; G06N 3/04

(52) U.S. Cl. CPC G16B 30/20; G16B 5/20; G16B 40/20; G06N 3/0454

(57) ABSTRACT

The invention generally relates to the field of protein sequences and of generation of functional protein sequences. More particularly, the invention concerns a method for generating functional protein sequences with generative adversarial networks. The described method for functional sequence generation comprises plurality of steps, each of which is crucial to ensure the high percentage of functional sequences in the final sequence set: selecting a plurality of existing protein sequences to define the approximate sequence space for the later generated synthetic sequences, processing the selected protein sequences, approximating the unknown true distribution of amino acids of the pre-processed sequences using a variation of generative adversarial networks, obtaining protein sequences from the approximated distribution, processing of the obtained protein sequences. The described method provides a resource (e.g. time, cost) efficient way of producing synthetic protein sequences which have a high probability of being functional experimentally.

### FIELD OF THE INVENTION

The invention generally relates to the field of protein sequences and of generation of functional protein sequences. More particularly, the invention concerns a method for generating functional protein sequences with generative adversarial networks.

### BACKGROUND OF THE INVENTION

Proteins are molecules consisting of chains of amino acids which can fold in 3-dimensional space to form molecular machines for catalysis of various chemical reactions.

Recombinant proteins were found to be extremely useful and are frequently used in medical applications such as antibodies, vaccines and growth factors. Additionally, proteins which have catalytic properties (enzymes) are actively used in various industries, e.g. biofuel, food and chemical synthesis. With the 20 commonly occurring proteinogenic amino acids, a protein comprising 100 amino acids, for instance, can be made from up to 20¹⁰⁰ unique sequence variants, making the systematic exploration of protein variants extremely challenging. In such an astronomical sequence space, as little as 1 in 10⁷⁷ of the possible protein sequences fold into the requisite three-dimensional structures to carry out their biological functions (Keefe and Szostak 2001; Taverna and Goldstein 2002; Axe 2004). The use of standard random mutagenesis to navigate this protein fitness landscape (Romero and Arnold 2009a) is often inefficient, as protein fitness declines exponentially with each random mutation (Bloom et al. 2005; Guo, Choe, and Loeb 2004a). Hence, it is immensely difficult to find a desired, functional protein variant due to the large part of sequence space being non-functional or not folding correctly—a tiny fraction of sequence space that needs to be tested. Experimental screening techniques are also limited to testing only 10⁶⁻⁹ protein variants. Additionally, up to 70% of single amino acid substitutions result in a decline of protein activity and 50% are deleterious to protein function (Romero and Arnold 2009b; Bloom et al. 2006; Guo, Choe, and Loeb 2004b; Rennell et al. 1991; Axe, Foster, and Fersht 1998; Shafikhani et al. 1997; Rockah-Shmuel, Tóth-Petróczy, and Tawfik 2015; Sarkisyan et al. 2016). In contrast, recombination of naturally occurring homologous proteins generates functional proteins with many mutations in a single step (Voigt et al. 2002; Hansson et al. 1999; Crameri et al. 1998). For instance, β-lactamase containing 75 mutations derived from a recombination library is 10¹⁶ times more likely to fold than one containing 75 random mutations (Drummond et al. 2005). However, these strategies are strongly limited by the number of available parent molecules.

Recent deep learning approaches have demonstrated great potential in capturing the structural, evolutionary, and biophysical information found in natural protein sequences, enabling inference of protein properties and prediction of protein function (Alley et al., n.d.). Machine learning models of complex epistatic sequence relationships can predict protein variant activity-based merely on existing sequences (Riesselman, Ingraham, and Marks 2018). Yet, despite the promise that these computational methods hold for navigating the fitness landscapes (Romero, Krause, and Arnold 2013; Yang, Wu, and Arnold 2019), they have until now been used primarily for sequence inference-based function prediction using readily available data. Deep generative algorithms capable of producing protein sequences have been tested recently using autoregressive neural networks (WO2019097014). However, these methods do not ensure the correct folding or chemical activity of the generated proteins, making the whole procedure effectively as inefficient as currently used random experimental approaches.

Therefore, there is a need for a novel method that can efficiently produce experimentally active protein sequences.

### SUMMARY OF THE INVENTION

The invention generally relates to the field of protein sequences and of generation of functional protein sequences. More particularly, the invention concerns a method for generating functional protein sequences with generative adversarial networks.

The described method for functional sequence generation comprises plurality of steps, each of which is crucial to ensure the high percentage of functional sequences in the final produced sequence set: selecting a plurality of existing protein sequences to define the approximate sequence space for the later generated synthetic sequences 601, processing the selected protein sequences 602, approximating the unknown true distribution of amino acids of the pre-processed sequences using a variation of generative adversarial networks 603, obtaining protein sequences from the approximated distribution 604, processing of the obtained protein sequences 605.

The described method provides a cost (as well as other resources such as time and similar) effective way of producing synthetic protein sequences which have a high probability of being functional experimentally.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numerical. For purposes of clarity, not every component is labelled in every figure, nor is every component of each embodiment of the invention shown where illustration is not necessary to allow those of ordinary skill in the art to understand the invention. In the figures:

FIG. 1 illustrates a flowchart describing high level architecture of the generative adversarial network;

FIG. 2 illustrates a flowchart describing the architecture of the generator network;

FIG. 3 illustrates a flowchart describing the architecture of Resnet block in the generator network;

FIG. 4 illustrates a flowchart describing the architecture of the discriminator network;

FIG. 5 illustrates a flowchart describing the architecture of the Resnet block in the discriminator network;

FIG. 6 illustrates a flowchart describing the main steps involved in the invented method;

FIG. 7 illustrates a flowchart of the overall network architecture used in example 1;

FIG. 8 illustrates generated sequence identity to the nearest natural sequence throughout training in different timestamps;

FIG. 9 illustrates the losses of generator and discriminator during training period. Generator and Discriminator losses become relatively stable after initial phase and eventually reach plateau;

FIG. 10 illustrates the sequence variability expressed as Shannon entropies for generated and training sequences estimated from multiple-sequence alignment (MSA). Low Shannon entropy values represent highly conserved and thus functionally relevant positions, whereas high entropy indicates high amino acid diversity at a given position;

FIG. 11 illustrates the fact that the generative adversarial network learns evolutionary conserved and functionally relevant positions;

FIG. 12 illustrates the GAN's ability to recreate positional amino acid distribution shown as Pearson's correlation coefficient for generated and natural sequences estimated from multiple-sequence alignment. Positions with lower correlation coefficients matches positions with higher sequence variability. Only positions with number of gaps lower than 75% are represented, moving average;

FIG. 13 illustrates the amino acid pair association (Zm) matrices for Natural and Generated protein sequences. Positive values indicate larger distance in comparison to random sequences with the same amino acid frequency, i.e., an integer number indicates how many positions on average the amino acids are further apart than in a random sequence;

FIG. 14 illustrates the amino acid pair correlations of produced synthetic and selected training sequences. Every point on the map represents the correlation of frequencies amino acid pairs between two different data sets. High correlation denotes that the same pairwise long-distance amino acid interactions were found in both datasets;

FIG. 15 illustrates the protein sequence space, visualized by transforming a distance matrix derived from k-tuple measures of protein sequence alignment into a t-SNE embedding. Dot sizes represent the 70% identity cluster size for each representative;

FIG. 16 illustrates the CATH domain diversity generated throughout evolution of ProteinGAN. At every 1200 training steps, 64 sequences were sampled and searched for representative CATH domains (E-value<1 e-6). Inset: ProteinGAN generated novel domains not found in natural sequences, as comparison of natural and generated sequences to mutated random control sequences demonstrated that sequence generation was not a random process (Fisher's exact test p-value<8.2e-16);

FIG. 17 illustrates the comparison of sequence diversity between the produced synthetic sequences and the natural (training) MDH dataset. Generated sequences are grouped into more diverse clusters. Inset shows the ratio of number of clusters (Y-axis) at different sequence identity cut-offs (X-axis);

FIG. 18 illustrates the activity levels of synthetic MDH proteins, as well as natural MDH protein controls;

FIG. 19 illustrates the malate production levels of synthetic MDH proteins in comparison to natural MDH proteins.

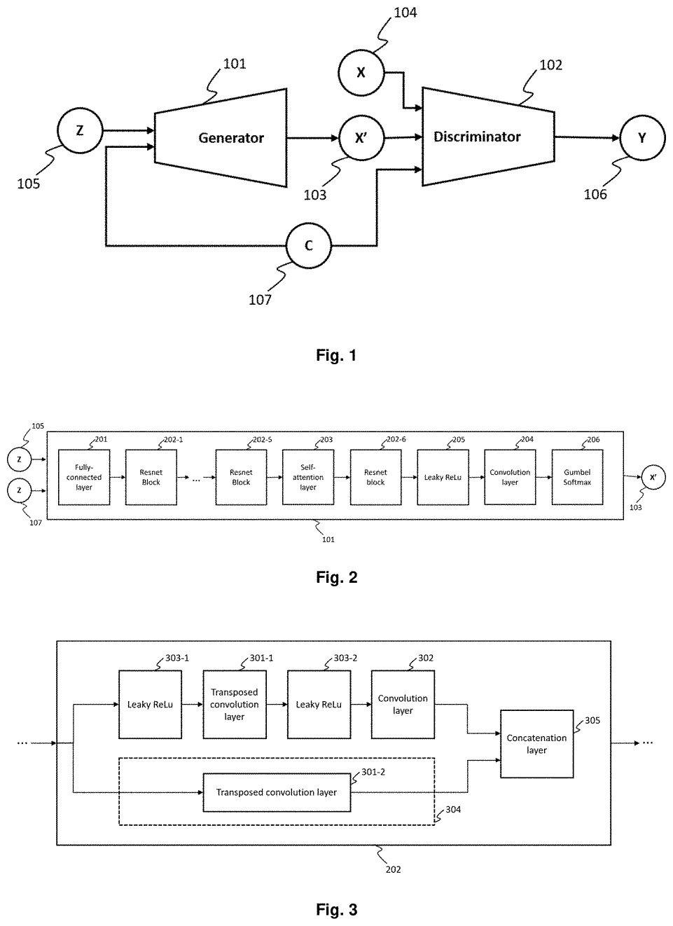

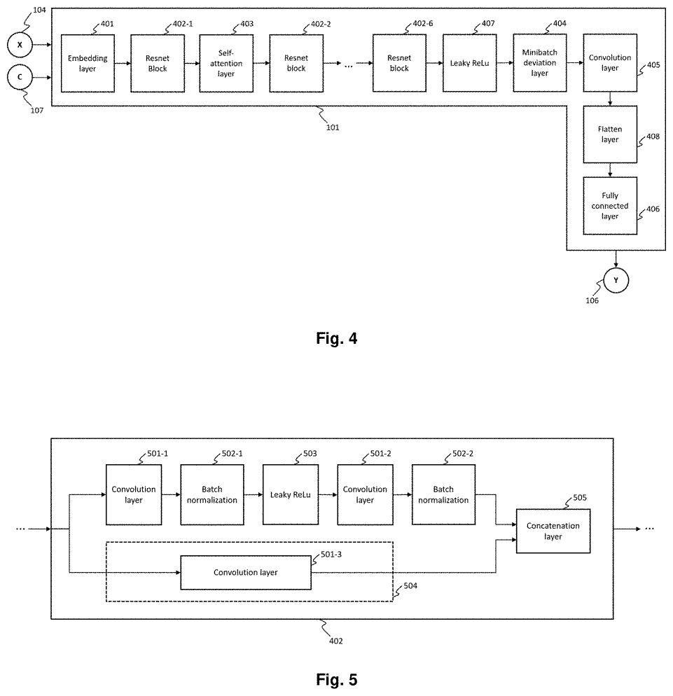

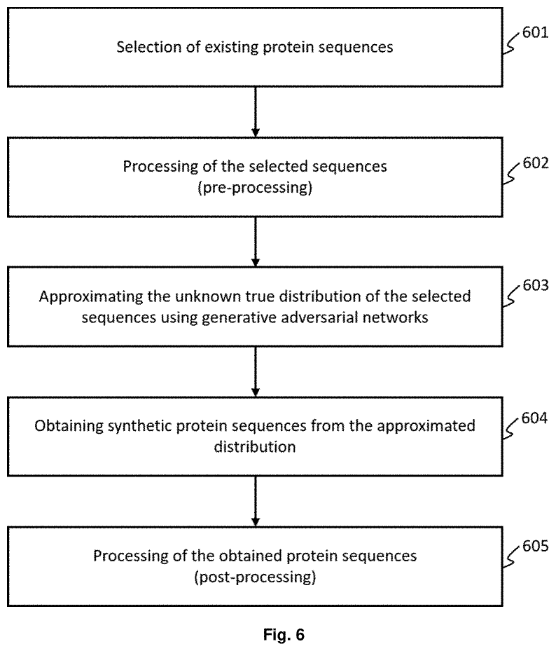

### DETAILED DESCRIPTION OF THE INVENTION

Reference will now be made in detail to exemplary embodiments of the invention. While the invention will be described in conjunction with the exemplary embodiments, it should be understood that they are not intended to limit the invention to these embodiments. On the contrary, the invention is intended to cover alternatives, modifications and equivalents, which may be included within the scope of the invention.

Throughout this disclosure, various aspects of this invention can be presented in a range format. It should be understood that the description in range format is merely for convenience and brevity and should not be construed as an inflexible limitation on the scope of the invention.

The described method for functional sequence generation comprises plurality of steps, each of which are crucial to ensure the high percentage of functional sequences in the final produced sequence set: selecting a plurality of existing protein sequences to define the approximate sequence space for the later generated synthetic sequences 601, processing the selected protein sequences 602, approximating the unknown true distribution of amino acids of the pre-processed sequences using a variation of generative adversarial networks 603, obtaining protein sequences from the approximated distribution 604, processing of the obtained protein sequences 605.

The described method provides a cost (as well as other resources such as time and similar) effective way of producing synthetic protein sequences which have a high chance of being functional experimentally.

### Definitions

To aid in understanding the invention, several terms are defined below.

Unless defined otherwise, all technical and scientific terms used herein have the same meaning as commonly understood by a person skilled in the art. Although any methods similar or equivalent to those described herein can be used in the practice or testing of the claims, the exemplary methods are described herein.

The terms “comprising”, “having”, “including”, and “containing” are to be construed as open-ended terms (i.e., meaning “including, but not limited to”) unless otherwise noted.

The term “bio-molecule” or “biomolecule” refers to a molecule that is generally found in a biological organism. Preferred biological molecules include biological macromolecules that are typically polymeric in nature being composed of multiple subunits (i.e., “biopolymers”). Typical bio-molecules include, but are not limited to molecules that share some structural features with naturally occurring polymers such as an RNAS (formed from nucleotide subunits), DNAs (formed from nucleotide Subunits), and polypeptides (formed from amino acid Subunits), including, e.g., RNAs, RNA analogues, DNAs, DNA analogues, polypeptides, polypeptide analogues, peptide nucleic acids (PNAS), combinations of RNA and DNA (e.g., chimeraplasty), or the like. Bio-molecules also include, e.g., lipids, carbohydrates, or other organic molecules that are made by one or more genetically encodable molecules (e.g., one or more enzymes or enzyme pathways) or the like.

The term “natural sequence” refers to amino acid sequences which are known from nature (e.g. a sequence derived from a gene such as a germline gene, or a sequence of a naturally occurring antibody). Accordingly, the term “artificial sequence” refers to amino acid sequences which are not known from nature.

The term “synthetic sequence” or “generated sequence” as used herein, refers to a protein sequences created by the described invention.

The term “sequence space” as used herein, refers to a space where all possible protein neighbours can be obtained by a series of single point mutations.

The term “neural network” or “network” as used herein, refers to a machine learning model that can be tuned (e.g. trained) based on inputs to approximate unknown functions. In particular, the term neural network can include a model of interconnected neurons that communicate and learn to approximate complex functions and generate outputs based on a plurality of inputs provided to the model. For instance, the term neural network includes one or more machine learning algorithms. In particular, the term neural network can include deep convolutional neural networks, such as a spatial transformer network. In addition, a neural network is an algorithm (or set of algorithms) that implements deep learning techniques that utilize the algorithm to model high-level abstractions in data.

The term “adversarial learning” refers to a machine learning algorithm (e.g. generative adversarial network) where opposing learning models are learned together. In particular, the term “adversarial learning” includes solving a plurality of learning tasks in the same model (e.g. in sequence or in parallel) while utilizing the roles and constraints across the tasks. In some embodiments, adversarial learning includes employing a minimax function (e.g. a minimax objective function) that both minimizes a first type of loss and maximizes a second type of loss. For example, the image composite system employs adversarial learning to minimize loss for generating warp parameters by a geometric prediction neural network and maximize discrimination of an adversarial discrimination neural network against non-realistic images generated by the geometric prediction neural network.

The term “motif” refers to a pattern of subunits in/or among biological molecules. For example, the motif can refer to a Subunit pattern of the unencoded biological molecule or to a Subunit pattern of an encoded representation of a biological molecule.

The terms “polypeptide” and “protein” are used interchangeably herein to refer to a polymer (or sequence) of amino acid residues. Typically, the polymer has at least about 30 amino acid residues, and usually at least about 50 amino acid residues. More typically, they contain at least about 100 amino acid residues. The terms apply to amino acid polymers in which one or more amino acid residues are analogues, derivatives or mimetics of corresponding naturally occurring amino acids, as well as to naturally occurring amino acid polymers. For example, polypeptides can be modified or derivatized, e.g., by the addition of carbohydrate residues to form glycoproteins. The terms “polypeptide” and “protein” include glycoproteins, as well as non-glycoproteins.

A “amino acid sequence” refers to the order and identity of the amino acids comprising a polypeptide or protein.

The term “screening” refers to the process in which one or more properties of one or more bio-molecule is determined. For example, typical screening processes include those in which one or more properties of one or more members of one or more libraries is/are determined.

The term “selection” refers to the process in which one or more bio-molecules are identified as having one or more properties of interest. Thus, for example, one can screen a library to determine one or more properties of one or more library members. If one or more of the library members is/are identified as possessing a property of interest, it is selected. Selection can include the isolation of a library member, but this is not necessary. Further, selection and screening can be, and often are, simultaneous.

The terms “subsequence” or “fragment” refers to any portion of an entire sequence of nucleic acids or amino acids.

The terms “library” or “population” refers to a collection of at least two different molecules and/or character Strings, such as nucleic acid sequences (e.g., genes, oligonucleotides, etc.) or expression products (e.g., enzymes) therefrom. A library or population generally includes a number of different molecules. For example, a library or population typically includes at least about 10 different molecules. Large libraries typically include at least about 100 different molecules, more typically at least about 1000 different molecules. For some applications, the library includes at least about 10000 or more different molecules.

The term “identity” (of proteins and polypeptides) with respect to amino acid sequences is used for a comparison of proteins chains. Calculations of “sequence identity” between two sequences are performed as follows. The sequences are aligned for optimal comparison purposes (e.g., gaps can be introduced in one or both of a first and a second amino acid sequence for optimal alignment and non-homologous sequences can be disregarded for comparison purposes). The optimal alignment is determined as the best score using the “ssearch36” program in the FASTA36 software package (http://faculty.virginia.edu/wrpearson/fasta/) with a Blosum50 scoring matrix with a gap-open penalty of −10, and a gap-extension penalty of −2. The amino acid residues at corresponding amino acid positions are then compared. When a position in the first sequence is occupied by the same amino acid residue at the corresponding position in the second sequence, then the molecules are identical at that position. The percent identity between the two sequences is a function of the number of identical positions shared by the sequences.

The term “functional protein” or “functional sequence” refers to a protein that is in a form in which it exhibits a property and/or activity by which it is characterized.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemagglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by, the trained artisan, and are contemplated to be within the scope of this definition.

The term “data augmentation” as used herein, refers to a strategy that enables to artificially increase the diversity of data available for training, without physically collecting data samples. Examples of data augmentation techniques for images are cropping, padding, and horizontal flipping.

The term “dataset” as used herein, refers to a collection of items that are used for training or evaluating neural networks.

The term “true distribution” as used herein, refers to a distribution which contains all real elements including the elements from a dataset.

The term “blocks” as used herein, in the context of neural networks refers to a group of architectural neural network components that are combined together and reused.

The term “differentiable discrete approximation” as used herein, refers to a function that converts continuous values to a discrete space and this function is differentiable.

The term “vocabulary size” as used herein, refers to a number of unique tokens used to construct items in the dataset. These tokens are discrete (e.g. amino acids).

The term “training step” as used herein, refers to neural network optimization cycle that process a set of elements where the size of set is equal to batch size.

### Selection and Pre-Processing of Existing Sequences

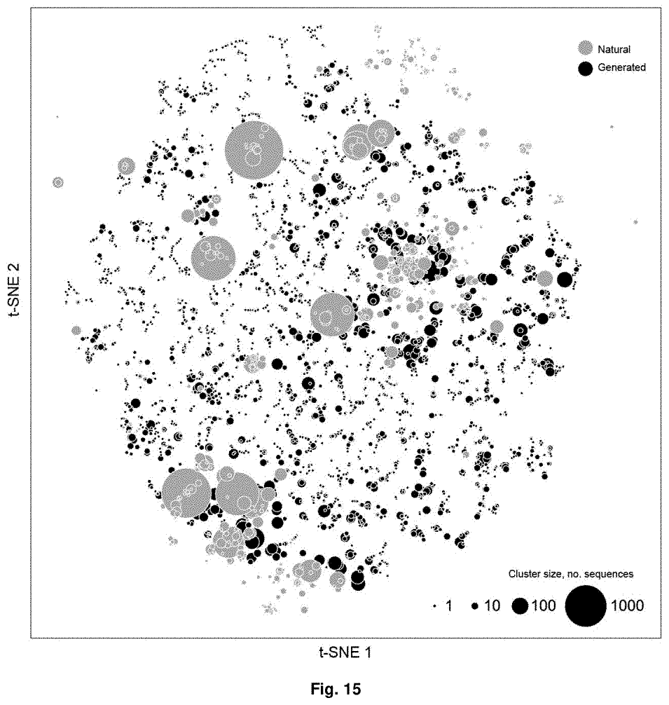

In one set of embodiments, existing sequences may be specifically selected for the training of generative adversarial network. Initial selection of sequence set(s) is an important procedure for several reasons: the selected sequences will define the sequence space in which the produced functional synthetic sequences will appear (i), the characteristics of selected sequences will define the unknown distribution that may be approximated in the generative adversarial network learning step (ii) and in turn may define some of the characteristics of produced synthetic sequences. An experimental, data driven example is shown in FIG. 15. In this figure, natural and synthetic (output from the described method) are displayed, wherein the distances between different clusters are comparable to cluster sequence-wise similarities and other similar characteristics. As described previously, the synthetic sequences appear in the approximate boundaries set by the natural clusters, making the first step of the method—selection of the sequences—extremely important.

For example, in order to explore the sequence space containing functional variants of Glycerol-3-phosphate dehydrogenase, one may choose the training sequences that fall into that area of sequence space. Such sequences may be homologs of Glycerol-3-phosphate dehydrogenase. These functional sequences may be acquired from public databases, metagenomics screening, random mutagenesis screening, rational variant screening or other sources. The collected sequenced dataset may then be further modified.

The selected sequences may then be processed by bioinformatic algorithms. This step is of high importance as unprocessed sequences used in the training of generative adversarial network have a high chance of yielding non-functional and/or insoluble final produced synthetic protein sequences.

In one set of embodiments, the pre-processing of the selected protein sequences may include the filtering of sequences using defined criteria, such as sequence origin, similarity, diversity, sequence cluster sizes, structural similarity, presence of domains, function or functional characteristics, statistical properties (e.g. amino acid frequencies or presence of non-canonical amino acids, working conditions), physicochemical properties, or other similar techniques.

In another set of embodiments, the pre-processing of the selected protein sequences may include modifying the selected sequences. Modification of the selected sequences may be sequence up sampling using techniques such as domain and/or motif shuffling, performing circular permutation, introducing mutations to sequences, including additional sequence fragments (e.g. linkers, tags, motifs), using only defined parts of the sequences (e.g. domains, motifs), combining different sequences into one sequence entity or similar techniques.

Data augmentation techniques may be used to increase the number and/or diversity of selected sequences (e.g. in events when the selected sequence number is too low to be used with described method), such as introduction of invariant transformations, interpolation, introduction of noise or other techniques.

In yet another set of embodiments, the selected sequences may be converted into different representations such as one-hot encoding, sequence embeddings (conversion of sequences into numerical values) or other. These different representations may also be modified by adding or removing quantitative or qualitative information, by techniques such as concatenation, input multiplication or other.

### Generative Adversarial Network Architecture for Protein Sequence Generation

The selected and pre-processed sequences may then be used as training (example) sequences for generative adversarial networks. The architecture of generative adversarial networks required for functional protein sequence generation is described further.

The reference numbers in the following paragraphs should be understood as an example, and other similar variants of architecture may also be viable.

Generative adversarial network architecture is comprised of two neural networks: the generator network 101 and discriminator network 102. The function of generator network 101 is to produce outputs 103 that appear to be drawn from the true distribution of the dataset 104 without having access to items of the distribution during the training. Discriminator network 102 receives inputs 104 from the dataset and generator 101 and is tasked with distinguishing generated items from the real ones. In general case, the training of generative adversarial network consists of: randomly choosing points from selected distribution 105 and generating samples 103 using the generator 101 (i), randomly choosing elements from dataset 104 (ii), using the discriminator 102 to get scores 106 for the generated 103 and dataset samples 104 using discriminator scores 106 to optimize the discriminator network 102 and the generator network 101 independently (iv), repeating described i-iv steps until generated samples are of desired quality or discriminator network 102 is unable to distinguish generated samples 103 from the real ones 104. The discriminator and generator networks may also be provided with additional information 107 making the overall generative adversarial network conditioned on the provided additional information.

In one set of embodiments, the generative adversarial network architecture consists of two networks—generator 101 and discriminator 102—each of which may contain a number of building blocks such as Resnet blocks 201, 401 (He et al. 2015). As an alternative to Resnet blocks, convolutional layers, fully connected layers, multi-head attention mechanism (Vaswani et al. 2017) or other architectural building blocks may be used.

In another set of embodiments, the generator input 105 may be a vector that is drawn from any known distribution such as uniform or normal. Generator network may contain one or more fully connected 201, convolutional layers before ResNet blocks 202 (e.g. 6 Resnet Blocks 202-[1-6]) to transform an input 105 to required dimensions. The generator network may have one or more self-attention (Zhang et al. 2018) layers 203. The generator network may contain one or more fully connected or convolutional layers 204 with non-linear activation function such as leaky ReLu 205, ReLu and others to produce an output 103 of desired dimensions. The output may be passed through an non-linear activation function (for example, Tahn, Softmax and others) as well as a differentiable discrete approximation of the output such as Gumbel-Softmax 206 or REINFORCE (Williams 1992). Additionally, during the training, the generator network may also be provided with additional information 107, such as a class label, which may be encoded using embeddings, one-hot encoding or transformed in other ways and then concatenated with one or more of the layers in the generator network.

In another set of embodiments, each Resnet block in generator 201 may consist of 1 to 10 transposed convolution layers 301 (e.g. 2 transposed convolution layers 301-[1-2]) and 1 to 10 convolution layers 302 with the filter size (1 to 100)×(1 to100). Convolution layers may contain dilation rates ranging from 1 to 10000. The blocks may contain a plurality of regularization layers such as batch normalization (Ioffe and Szegedy 2015), instance normalization (Ulyanov, Vedaldi, and Lempitsky 2016) and others. Moreover, blocks may also contain various activation functions such as leaky ReLu 303 (Maas 2013) (e.g. 2 leaky ReLu activations 303-[1-2]), ReLu (Nair and Hinton 2010) and others. Blocks also may contain 1-10 skip connections 304 which may be concatenated 305 with other parts of the block. To increase the dimensions of the layer output instead of transposed convolution layer 301, nearest-neighbour interpolation, sub-pixel shuffle (Shi et al. 2016) or other techniques may be used.

In another set of embodiments, the input 104 to discriminator network may be one-hot encoded with vocabulary size ranging from 10 to 10 000 or similar. Alternatively, the input may be encoded using amino acid embeddings or physicochemical attributes. Discriminator network may contain one or more embedding 401, convolution or fully connected layers before Resnet blocks 402 (e.g. 6 Resnet blocks 402-[1-6]) to transform the input 104. Moreover, it may contain one or more self-attention layers 403. Discriminator network may contain a layer to maintain high variety between generated sequences such as minibatch standard deviation layer 404 as described in (Karras et al. 2017). Discriminator network may contain one or more convolution 405, fully connected 406 layers, or global average poolings with non-linear activation functions such as leaky ReLu 407, ReLu and others to produce an output of desired dimensions. Some of layers may be flattened using Flatten layers 408. The final outcome of the discriminator may be passed through a non-linear activation function such as Softmax, Tanh or other.

In another set of embodiments, each Resnet block in the discriminator may contain 1, 2, 3, 4, 5, 6, 7, 8, 9 or 10 convolution 501 (e.g. 3 convolution layers 501-[1-3]) and/or fully connected layers with filter the size of (1 to 100)×(1 to 100). Convolution layers may contain dilation rates (1 to 10000). Blocks may contain a plurality of regularization layers such as batch normalization 502 (e.g. 2 batch normalization layers 502-[1-2]), instance normalization and others. Blocks may also contain various non-linear activation functions such as leaky ReLu 503, ReLu and others. Blocks also may contain 1-10 skip connections 504 which may be concatenated 505 with other part of block. During the training, the discriminator network may also be provided with additional information 107 along with the pre-processed training sequences, such as a class label, which may be encoded using embeddings, one-hot encoding or transformed in other ways and then concatenated with one or more of the layers in the discriminator network.

In another set of embodiments, for the network loss, non-saturating (Goodfellow et al. 2014), non-saturating with R1 regularization (Mescheder, Geiger, and Nowozin 2018), hinge (Tran, Ranganath, and Blei 2017; Lim and Ye 2017; Miyato et al. 2018), hinge with relativistic average (Jolicoeur-Martineau 2018), Wasserstein (Arjovsky, Chintala, and Bottou 2017) and Wasserstein with gradient penalty (Gulrajani et al. 2017) or other functions may be used. To ensure Lipschitz constraint spectral normalization (Miyato et al. 2018), gradient penalty (Gulrajani et al. 2017) or other techniques may be used.

In another set of embodiments, the dimensions of generated outputs depend on the maximum length of the sequence required to be generated and the type of discriminator network encoding used. For example, for maximum sequence length of 400 amino acids and one-hot encoding with vocabulary size of 21, the dimensions of generated output would be 400×21.

Depending on the chosen generated output dimensions sequences selected for training may be further filtered to remove sequences containing more amino acids than the output dimensions allow. For example, if the dimensions of generated outputs are 400×21, sequence dataset may be filtered to remove sequences that are over 400 amino acids. The dataset may also be clustered into clusters with specific identities. For example, this may be achieved by using clustering tools such as mmseq2 or other. The clustering allows to balance the generative adversarial network training process, which is important in order to achieve synthetic functional sequence variation. Sequences based on their cluster size may be grouped into buckets of various sizes (1,2,3,5,10,20,30, etc.). Then the upsampling factor is determined by dividing maximum bucket size by cluster bucket size for all buckets. This factor is used to upsample under represented clusters during the training. A part of the dataset may be selected randomly or rationally and taken out of the training dataset. Such sequences may then act as validation sequences that the network will not see during the training but can later be used for network performance analysis purposes.

In another set of embodiments, to optimize neural network weights, ADAM optimizer (Kingma and Ba 2014), Stochastic Gradient Descent (Kiefer and Wolfowitz 1952), RMSProp (Graves 2013) and other optimizers may be used for the generator and discriminator networks. The learning rate may be gradually decreased for both generator and discriminator to increase training stability and aid the convergence. For example, the gradual decrease for the learning rate may be from 1 e-3 to 5e-5. Ratio between generator and discriminator training steps may be 1:1 1:2, 1:5 or other.

In yet another set of embodiments, to normalize the data cluster sizes during the generative adversarial network training, under-represented sequence clusters may be dynamically up-sampled. This may be achieved by up-sampling under-represented clusters (duplicating sequences inside of the cluster) by up-sampling factor that was calculated at the earlier stages. This process may be repeated throughout the generative adversarial network training in order to preserve the sequence variation. Sequences may be padded with special character dynamically to denote absence of amino acid. This may be used to pad shorter sequences if constructed network contains layers which require fixed size input such as fully connected. Sequences may be padded from the left, right or both sides. Padding is removed from generated sequences when final output is produced (for example, when one-hot encoded sequences are converted to sequences of single letter amino acids).

In yet another set of embodiments, in order to track the network's performance, the generated data should be evaluated throughout the training process. For example, every 1200 steps the generated sequences may be automatically aligned with the training and validation dataset sequences using BLAST or similar algorithms. To further exemplify, the periodically generated sequence during training procedure may also be subjected to calculation of blosum45, e-value and identity scores.

In yet another set of embodiments, after the training of generative adversarial network is done, in order to obtain protein sequences from learned distribution, random points are selected from the distribution that was used during training. To increase the quality of the generated examples, the standard deviation of used distribution may be reduced at the expense of sample variety. These points then are feed-forwarded through the trained generator to obtain generated representation of the sequence drawn from the determined true distribution that was learned during the training procedure. Obtained representation (e.g. one-hot encoded or embeddings) is then converted to sequences of amino acids and any gaps at the beginning or end of the sequences are removed.

### Processing of the Obtained Synthetic Protein Sequences

The synthetic protein sequences obtained by the generative adversarial network determined distribution may be subjected to further processing (post-processing) using bioinformatic techniques. This step is of great importance as it dramatically increases the probability of finding sequences that will yield experimentally functional proteins.

In one set of embodiments, the post-processing may incorporate computational filtering of obtained synthetic sequences. Such filtering procedure may be used to rank the obtained synthetic sequences by a defined criterion, such as discriminator score, generated qualitative or quantitative descriptors, scores or labels predicted by other models (e.g. machine learning models, quantitative structure-property relationship models, structural or molecular dynamics models) or other.

In another set of embodiments, the post-processing of synthetic sequences may be the modification of those sequences, such as providing stabilizing mutations, linker sequences, protein tags, combining the sequences with other protein sequences or other.

### Usage of the Produced Functional Protein Library

The output of the described method—a highly functional protein sequence library—may be then used in multiple applications such as experimental protein screening, data augmentation or other. The functional sequence library may be physically built by gene or protein synthesis methods. Then, the physical library may be screened experimentally using standard methods such as in-vitro/in-vivo protein expression and characteristic measurement, droplet microfluidics, or other. The screening may target a wide range of characteristics, such as the type of chemical reaction produced by protein variants, the activity level, thermostability, solubility or other. An example of the functional protein library generation and experimental screening is described in Example 1. The functional sequence library produced by the described invention may also be used for data augmentation purposes. In such cases, the method is used to enrich sequence set used by other machine learning algorithms with additional sequences produced by the described invention. Examples of such algorithms may be predicting optimal enzyme catalytic temperature, predicting secondary structure of protein or other.

### Examples

Hereafter, the present invention is described in greater detail with reference to the examples, although the technical scope of the present invention is not limited to the following examples.

### Example 1. Production of Functional Synthetic Malate Dehydrogenase Sequences

This is an example of the production of functional malate dehydrogenase (E.C. 1.1.1.37) synthetic protein sequences using the described invention. The goal of this example is to show how every step of the method may be executed.

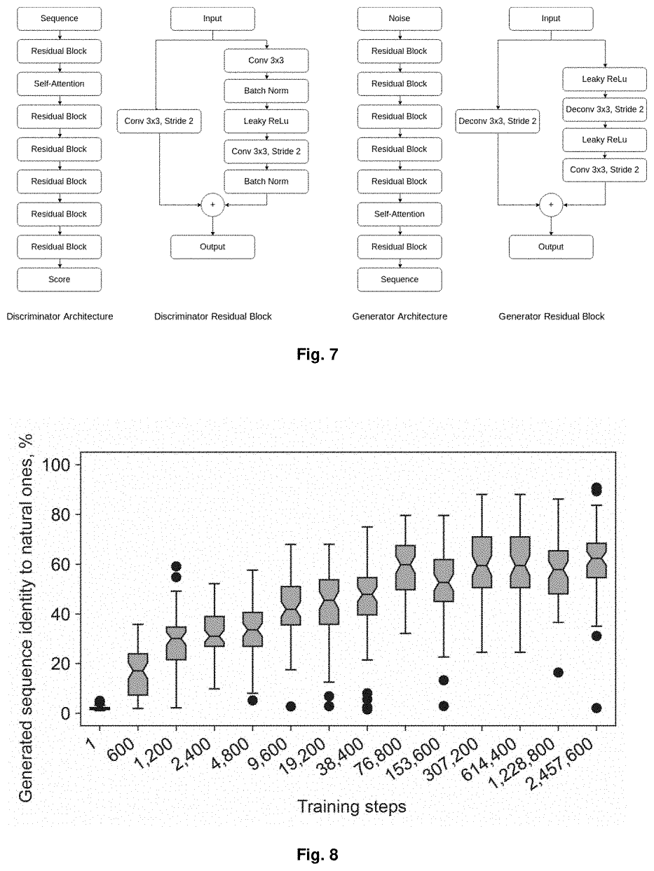

In this example, the generative adversarial network architecture consisted of two networks—discriminator and generator—each of which used ResNet blocks. The flowchart of the overall generative adversarial network architecture used in this example can be seen in FIG. 7. Each block in the discriminator contained 3 convolution layers with filter size of 3×3, 2 batch normalization layers and leaky ReLU activations. The generator residual blocks consisted of two transposed convolution layers, one convolution layer with the same filter size of 3×3 and leaky ReLU activations. Each network had one self-attention layer. Transposed convolution technique was chosen for up-sampling as it yielded the best results experimentally. For loss, non-saturating loss with R1 regularization was used. To ensure training stability spectral normalization was implemented in all layers.

The input to the discriminator was one-hot encoded with vocabulary size 21 (20 canonical amino acids and a sign that denoted space at the beginning or end of the sequence). The generator input was a vector of 128 values that were drawn from a random distribution with mean 0 and standard deviation of 0.5, except that values whose magnitude was more than 2 standard deviations away from the mean were re-sampled. The dimensions of generated outputs were 512×21 wherein some of the positions denoted spaces.

In this example, bacterial malate dehydrogenase (MDH) sequences were collected from public protein sequence database Uniprot. Sequences longer than 512 amino acids or containing non-canonical amino acids were filtered out. The final dataset consisted of 16898 sequences which were clustered into 70% identity clusters using MMseq2 tool (Steinegger and Soding 2017) for balancing the dataset during the training process. 20% of the clusters with less than 3 sequences were randomly selected for validation (192 sequences) and the rest of the dataset was used for training (16706 sequences). Eight representative, natural MDH sequences from the training dataset is provided (SEQ ID NO:1-SEQ ID NO:8).

The ratio between generator and discriminator training steps was selected 1:1. ADAM algorithm was used to optimize both networks. Throughout the training, the learning rate was gradually decreased from 1 e-3 to 5e-5 for both generator and discriminator. To avoid bias towards sequences with large number of homologues, smaller clusters were dynamically up-sampled during the training. In order to track the performance, along with GAN losses, generated data was constantly evaluated. Without halting the training process, every 1200 training steps generated sequences were automatically aligned with the training and validation datasets using BLAST (FIG. 8). The training took 210 hours (˜9 days) on NVIDIA Tesla P100 (16 GB).

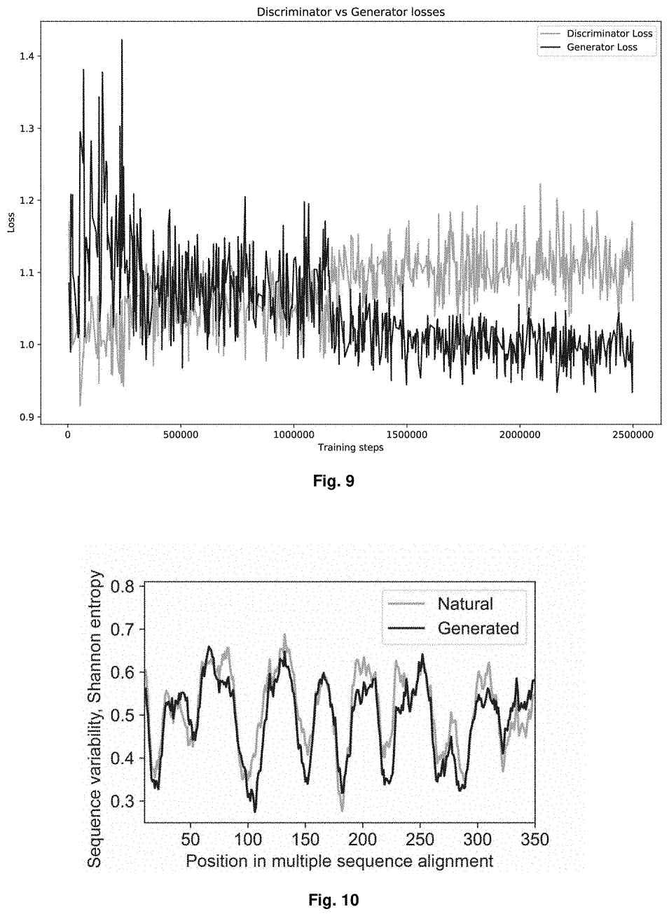

After 2.5M training steps, at which training was terminated, the mean sequence identities between the generated and natural sequence sets had reached a plateau (median seq. identity to the closest natural sequences was 61.3%, (FIG. 9). Following the initial quality assessment, 20 000 sequences were generated for further analysis of the trained network.

Neural network's ability to capture which positions in the sequence are conserved and which are variable by computing Shannon entropies for each position in the network-generated and natural sequences (FIG. 10).

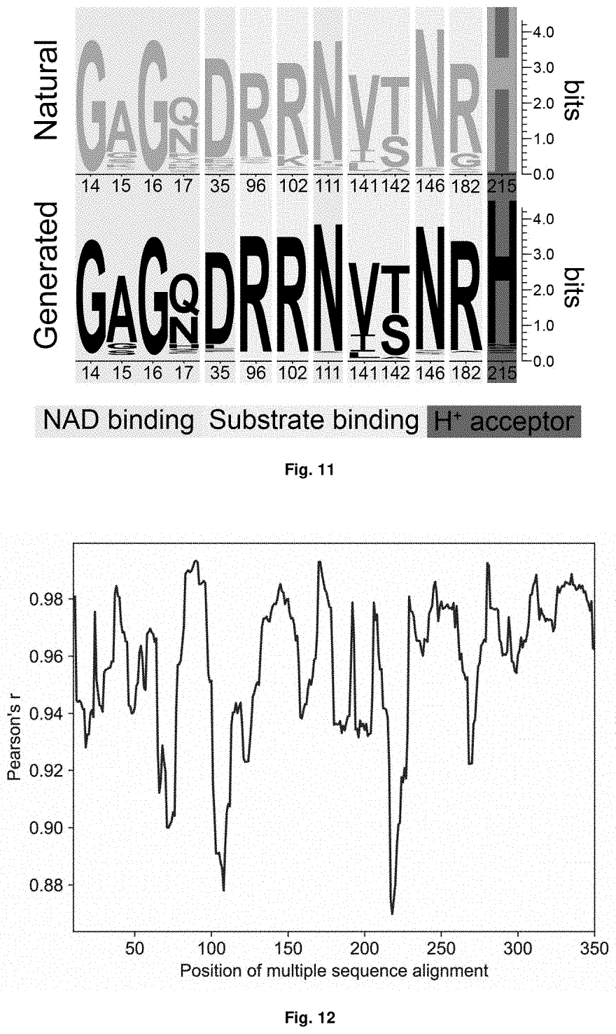

The positional variability in generated sequences was highly similar to that in natural sequences, with peaks (high entropy) and valleys (low entropy) appearing at similar positions in the sequence alignment. Indeed, there is an almost perfect correlation between the entropy values of generated and natural sequences (Pearson's r=0.89, P-value<1 e-16). The generated sequences preserved substrate-binding and catalytic residues by learning the conserved amino acid positions that are critical for catalysis (FIG. 11).

Further comparative analysis of generated and natural sequences showed that even in highly variable sequence regions, the frequencies of individual amino acids were perfectly correlated (Pearson's r=0.96, P-value<1e-16, FIG. 12).

As a result, our specific generative network architecture inferred the specific physicochemical signatures in the variable sequence regions, which are unique for every homologue, yet complementarity add up to the same physicochemical signature of the individual sequence. For instance, despite the high sequence diversity, the fractions of hydrophobic, aromatic, charged and cysteine-containing residues were the same in generated sequences (Wilcoxon rank sum test P-value>0.05) as in natural ones. Apart from the differences (P-value=7e-5; 1 e-28, respectively) in hydrophilic and polar uncharged residues, the network has learned the overall amino acid patterns of similar evolutionary and physicochemical context (Table 1).

TABLE 1
Physicochemical properties of amino acids.

| Amino acids | Statistic | p-value | Properties |
|---|---|---|---|
| W | −44.5535 | 0 | — |
| T | −32.6757 | 3.45E−234 | — |
| N | −31.0134 | 3.55E−211 | — |
| P | 5.414836 | 6.13E−08 | — |
| F | 36.10193 | 2.12E−285 | — |
| A | −7.00421 | 2.48E−12 | — |
| G | 2.373175 | 0.017636 | — |
| I | 10.88373 | 1.38E−27 | — |
| L | 24.0913 | 3.08E−128 | — |
| H | 0.883687 | 0.376865 | — |
| R | 16.52561 | 2.40E−61 | — |
| M | 2.927137 | 0.003421 | — |
| V | −37.0289 | 3.93E−300 | — |
| E | −17.0094 | 7.00E−65 | — |
| Y | −2.82555 | 0.00472 | — |
| V, I, L, F, | −0.64435 | 0.519345 | Hydrophobic |
| W, Y, M | | | |
| S, T, H, N, | −3.965 | 7.34E−05 | Hydrophilic |
| Q, E, D, K, R | | | |
| F, W, Y, H | −1.00434 | 0.315217 | Aromatic |
| P, G, A, S | 7.263205 | 3.78E−13 | Small |
| K, R, H | 5.612656 | 1.99E−08 | Positive |
| D, E | −22.965 | 1.04E−116 | Negative |
| V, I, L, M | −2.04672 | 0.040685 | Aliphatic |
| S, C, T, M | −0.12037 | 0.904194 | Hydroxyl |
| S, T, C, M, N, Q | −11.1188 | 1.02E−28 | Polar uncharged |
| H, K, R, E, D | 1.296764 | 0.194713 | Charged |

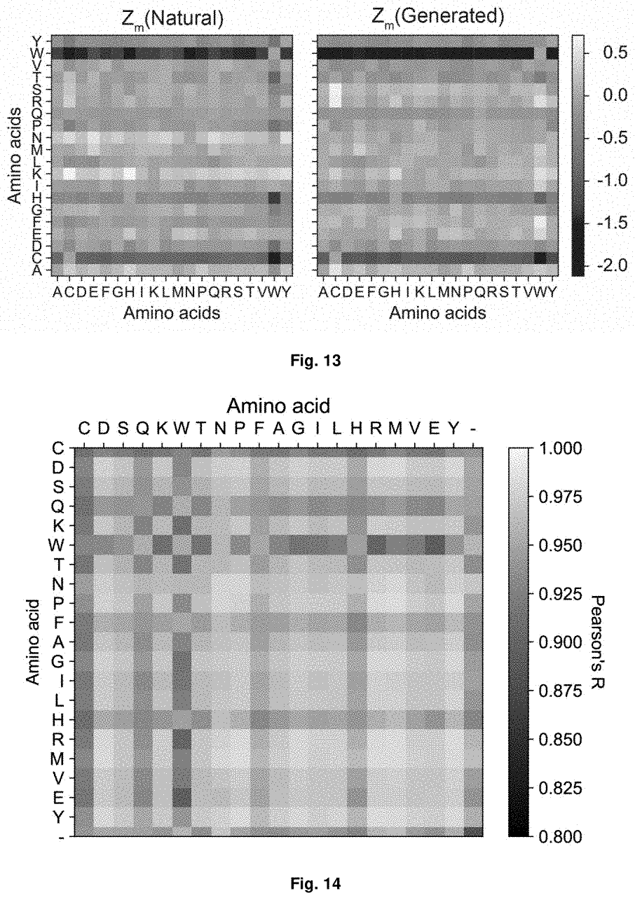

In proteins many amino acids pairs which are remote on the primary sequence are spatially close and interact in the 3D structure, ensuring the appropriate protein stability and function. We assessed whether the network was able to learn such local and global amino acid relationships by looking for long-distance pairwise amino acid relationships across the full length of the MDH sequences. For all the generated MDH sequences we calculated the amino acid association measures using the minimal proximity function Zₘ (Santoni et al. 2016). The function Zₘ(A,B) counts the closest average distance from each amino acid A to any amino acid B in the sequence and can be expressed as a matrix for all possible pairs (FIG. 13).

The matrices for the natural (training) and generated (synthetic) sequences were 88% similar with a slight difference for tryptophan as 22% of the natural sequences used did not have tryptophan. To further investigate the pairwise amino acid relationships, we calculated the correlation for all possible amino acid pairs for each combination of positions in multiple sequence alignments from natural and generated sequences. Overall, we found strong correlations between the natural and generated sequences (averaged Pearson's r=0.95, FIG. 14) demonstrating that the pairwise relationships are highly similar in both sets of sequences.

To expand on this, we inspected whether generated MDH sequences had the two main Pfam (Finn et al. 2014) domains identified (E-value<1e-10) in the natural MDH sequences (Ldh_1_N and Ldh_1_C). Indeed, we found that 98% of the generated sequences contained both signatures, with the rest containing only one of the domains. These results show that sequences generated by our invented method are of high quality and closely mimic natural MDH proteins, both in terms of amino acid distributions at individual sites, as well as in terms of long-distance relationships between pairs of amino acids present throughout the primary sequence of MDH family.

Next, we aimed to explore whether our trained network was also able to generalize the protein family and generate novel sequence diversity. First, we visualized generated and natural sequences sequence diversity using t-distributed stochastic neighbour embedding (t-SNE) dimension reduction (Maaten and Hinton 2008). As a majority of natural MDH sequences were highly similar (median pairwise identity 92%), they grouped into clusters and the generated sequences interpolated between the natural sequence clusters resembling a learned manifold of the MDH sequence space (FIG. 15).

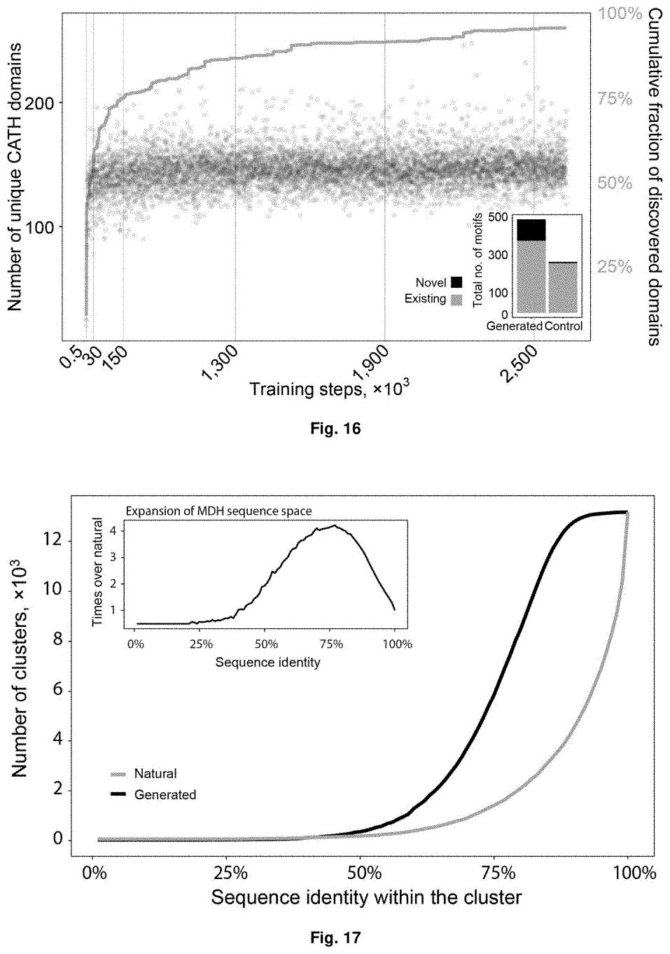

To assess whether generated diverse sequences would contain novel and functionally relevant biological properties, we performed a search of all OATH (Dawson et al. 2017) sequence models corresponding to all known 3D structural protein domains. First, we evaluated whether the network would evolve during the training by generating structural domain diversity over the training period (FIG. 16).

While the number of identified structural domains plateaued at the early stage of training (after 0.2M training steps) totalling in 79% of all identified domain space, structural CATH domains were discovered throughout the entire training process. In total, 119 novel structural sequence motifs (E-value<1e-6) were identified (inset of FIG. 16) in generated sequences that do not exist in natural bacterial malate dehydrogenase enzyme family. Afterwards, we have evaluated whether the generated structural domain diversity was not due to chance. To test this, as a control, we randomly introduced amino acid substitutions into the natural sequences, while preserving natural amino acid frequency distribution and the rate of mutations mimicking the natural sequence variability (inset of FIG. 16). The structural domain diversity was reduced by 38.9% in mutated natural sequences, 97.4% of mutated motifs were present in natural sequences demonstrating that random mutations do not produce biologically relevant sequence diversity (inset of FIG. 16), Fisher's exact test p-value<8.2e-16), Overall, over 95% of generated sequences were not more than 10% similar to each other (FIG. 17), in contrast to only 17% of the natural sequences with the same identity level, expanding up to 4 times (inset of FIG. 17) the currently known malate dehydrogenase family's sequence space.

As typical for up to 70% of all random amino mutations can be deleterious of variety of protein functions (Romero and Arnold 2009a; Bloom et al. 2006; Guo, Choe, and Loeb 2004a; Rennell et al. 1991; Axe, Foster, and Fersht 1998; Shafikhani et al. 1997; Rockah-Shmuel, Tóth-Petróczy, and Tawfik 2015; Sarkisyan et al. 2016), we wanted experimentally verify the generated natural-like diversity of novel homologous proteins were showing the malate dehydrogenase catalytic activity.

Before experimental testing, the obtained synthetic protein sequences were further subjected to post-processing in order to maximise the percentage of functional protein sequences in the generated set. The generated sequences were filtered via defined criteria: after assigning discriminator score to each of the sequence only the sequences from the first quartile of discriminator score were selected (i), synthetic sequences were aligned with the selected protein sequences used to train generative adversarial network and synthetic sequences with identity lower than 60% in comparison to the closest natural sequence are discarded (ii), the obtained synthetic sequences were scored and filtered by comparing them to the sequences selected for network's training in terms of their structural information (iii).

The structural comparison and evaluation of synthetic and natural sequences is a multi-step process. The most similar natural sequences which have solved protein structures were selected and assigned to every synthetic sequence. For every residue in a given structure, the number of other residues in close proximity to that residue were assigned. Then, every synthetic sequence was aligned with the initially assigned natural sequence. If an amino acid did not match in the natural and synthetic sequence pair alignment, the number of contacts associated to that residue position was added to a score. Finally, the synthetic sequences with the lowest scores were selected (variants which have their amino acid residue contacts changed the least)

Out of the produced synthetic sequences we have randomly selected 40 sequences with their pairwise sequence identity ranging from 64% to 98% and having from 6 to 45 amino acid substitutions compared to their closest neighbour in the natural MDH sequence space. The synthesized generated sequences were then recombinantly expressed in Escherichia coli, purified and in vitro tested for MDH catalytic activity.

In the following paragraph, detailed experimental conditions are provided. The sequences generated by invented method were synthesized, cloned into the pET21a expression vector and sequence-verified by Twist Bioscience. In addition to the enzyme sequence a C-terminal linker and four histidines (AAALEHHHH) were added, resulting in a deca-His-tag in the final construct which includes six histidines derived from the expression vector, to enable downstream affinity purification. The constructs were transformed into the BL21(DE3) E. coli expression strain. From the resulting transformation mixture 15 μl was used to inoculate 500 μl LB broth supplemented with 100 μg/ml carbenicillin. Cells were grown overnight at 32° C. in a 96 deep well plate with 700 rpm orbital shaking. Protein expression was achieved by diluting the overnight cultures 1:30 into 1 ml autoinduction TB including trace elements (Formedium, UK) supplemented with 100 μg/ml carbenicillin and grown for 4 h in 37° C., followed by overnight growth at 18° C. and 700 rpm shaking. Cells were collected by centrifugation and the cell pellets frozen in −80° C. overnight. To purify the recombinant proteins, cells were thawed, resuspended in 200 μl lysis buffer (50 mM HEPES pH 7.4, 5% glycerol, 300 mM NaCl, 0.5 mM TCEP, 0.5 mg/ml lysozyme, 10 U/ml DNasel, 2 mM MgCl₂), and incubated for 30 min at room temperature. To improve lysis triton-X-100 was added to a final concentration of 0.125% (v/v), and the cells were frozen in −80° C. for 30 min. After thawing in room temperature water bath, the lysates were spun down for 10 min in 3000×g to remove cell debris, and the supernatants were transferred to a new 96-well plate with 50 μl Talon resin in each well (Takara Bio, Japan). Unspecific binding of proteins to the resin was reduced by adding imidazole to a final concentration of 10 mM in each well. The plate was incubated at room temperature for 30 min with 400 rpm shaking, after which the lysates with the beads were transferred to a 96-well filter plate (Thermo Scientific, USA, Nunc 96-well filter plates) placed over a 96-well collection plate, and centrifuged for 1 min at 500×g in a swing-out centrifuge. The resin was washed three times with 200 μl wash buffer (50 mM HEPES pH 7.4, 5% glycerol, 300 mM NaCl, 0.5 mM TCEP, 40 mM imidazole), and the proteins were eluted from the resin in two 50 μl fractions using elution buffer (50 mM HEPES pH 7.4, 5% glycerol, 300 mM NaCl, 0.5 mM TCEP, 250 mM imidazole). The two eluate fractions were combined and transferred to a 96-well desalting plate (Thermo Scientific, USA, Zeba Spin Desalting Plate, 7K MWCO) pre-equilibrated with sample buffer (50 mM HEPES pH 7.4, 5% glycerol, 300 mM NaCl, 0.5 mM TCEP). The plate was spun down 1000×g for 1 min, and collected proteins were analysed by SDS-PAGE followed by Coomassie staining. The soluble proteins were carried on for further characterisation. To test for malate dehydrogenase activity, an aliquot of purified protein was added to a reaction mixture containing 0.15 mM NADH, 0.2 mM oxaloacetic acid, 20 mM HEPES buffer (pH 7.4). Final reaction volume was 100 μl, the reaction was carried out at room temperature in a UV-transparent 96-well half-area plate (UV-Star Microplate, Greiner, Austria). Activity was measured in triplicates by following NADH oxidation to NAD+, with absorbance reading at 340 nm performed every 30 sec for 15 min in a BMG Labtech SPECTROstar Nano spectrophotometer. Un-specific oxidation of NADH was monitored in no-substrate controls, and these values were subtracted from the other samples. LC-MS/MS quantification was performed for selected active enzymes. The activity assay was performed as outlined above, in triplicates, with protein concentrations ranging between 10 and 250 nM. Reactions were terminated after 45 min by diluting the assay mixtures in water to 1 μg/ml starting concentration of oxaloacetate. For chromatographic separation a Zorbax Eclipse Plus C18 50 mm×2.1 mm×1.8 μm (Agilent) with a Nexera series HPLC (Shimadzu) were used. Mobile phase A was composed of H₂O (MiliQ HPLC grade) with 0.1% Formic acid (Sigma); mobile phase B was Methanol (Sigma) with 0.1% Formic acid (Sigma). The oven temperature was 40° C. The chromatographic gradient was set to consecutively increase from 0% to 100%, hold, decrease from 100% to 0% and hold, in 60 sec, 30 sec, 30 sec and 30 sec, respectively. The autosampler temperature was 15° C. and the injection volume was 0.5 μl with full loop injection. For MS quantification a QTRAP® 6500 System (Sciex) was used, operating in negative mode with Multiple Reaction Monitoring (MRM) parameters optimized for Malic acid based on published parameters (McCloskey and Ubhi 2014). Electrospray ionization parameters were optimized for 0.8 mL/min flow rate and were as follows: electrospray voltage of −4500 V, temperature of 500° C., curtain gas of 40, CAD gas set to Medium, and gas 1 and 2 of 50 and 50 psi, respectively. The instrument was mass calibrated with a mixture of polypropylene glycol (PPG) standards. The software Analyst 1.7 (Sciex) and MultiQuant 3 (Sciex) was used for analysis and quantitation of results, respectively.

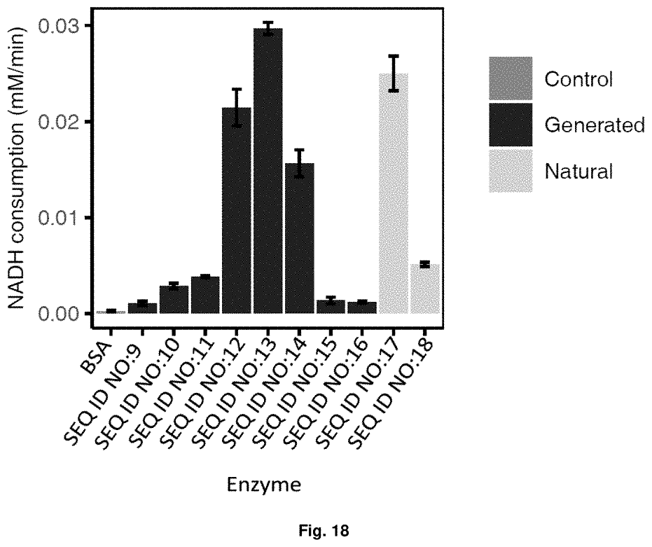

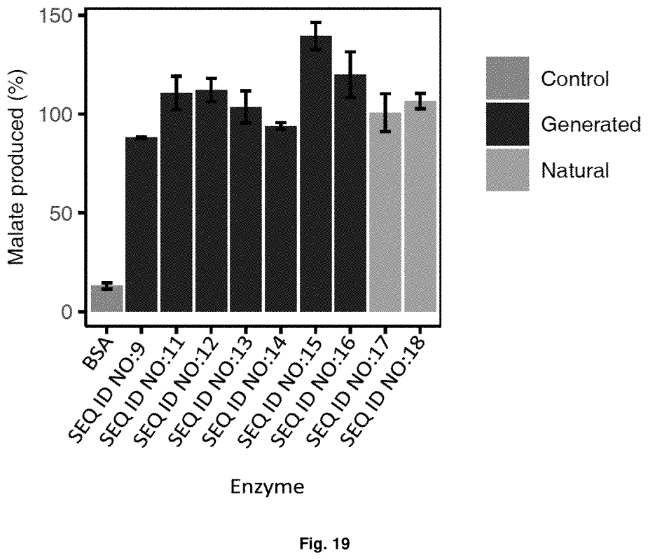

Ten of these 40 protein variants (25%) were expressed at high levels and were present in the soluble fraction after cell lysis, indicating protein folded conformation. This is indeed a high success rate considering that even when expressing natural enzymes in E. coli in systematic studies the soluble enzyme fraction can be as little as 20% (Huang et al. 2015; Bastard et al. 2017). The 10 soluble proteins were purified using affinity chromatography and assessed for malate dehydrogenase activity by fluorescently monitoring NADH consumption. 8 of 10 (80%) soluble enzymes, including the variant with 45 amino acid substitutions, showed robust catalytic activity (SEQ ID NO:9-SEQ ID NO:16, FIG. 18) with similar kinetics as wild-type sequences (SEQ ID NO:17 and SEQ ID NO: 18, FIG. 18). To confirm the specificity of the reaction, we monitored the product formation using LC-MS/MS operating in selected reaction monitoring mode. We confirmed oxaloacetate to malate formation (SEQ ID NO:9-SEQ ID NO:16, FIG. 19) with a comparable reaction yields as wild-type MDH analogues (SEQ ID NO:17-SEQ ID NO:18, FIG. 19).

To conclude, our provided experimental example demonstrates that our multi-step method for functional protein sequence generation confidently captures the numerous properties of natural proteins, such as sequence motifs, position-specific amino acid composition and long-range amino acid interactions, while also allowing the generation of catalytically active, functional and diverse sequences. We have experimentally confirmed the robust enzymatic activity in 80% of soluble generated enzymes. The invented method thus enables large jumps to unexplored sections of sequence space allowing sampling of highly diverse novel functional proteins within the learned biological constraints of the enzyme family in a cost and resource effective manner.

### REFERENCES

1. Alley, Ethan C., Grigory Khimulya, Surojit Biswas, Mohammed AlQuraishi, and George M. Church. n.d. “Unified Rational Protein Engineering with Sequence-Only Deep Representation Learning.” https://doi.org/10.1101/589333.
2. Arjovsky, Martin, Soumith Chintala, and Léon Bottou. 2017. “Wasserstein GAN.” http://arxiv.org/abs/1701.07875.
3. Axe, Douglas D. 2004. “Estimating the Prevalence of Protein Sequences Adopting Functional Enzyme Folds.” Journal of Molecular Biology 341 (5): 1295-1315.
4. Axe, Douglas D., Nicholas W. Foster, and Alan R. Fersht. 1998. “A Search for Single Substitutions That Eliminate Enzymatic Function in a Bacterial Ribonuclease†.” Biochemistry. https://doi.org/10.1021/bi9804028.
5. Bastard, Karine, Alain Perret, Aline Mariage, Thomas Bessonnet, Agnès Pinet-Turpault, Jean-Louis Petit, Ekaterina Darii, et al. 2017. “Parallel Evolution of Non-Homologous Isofunctional Enzymes in Methionine Biosynthesis.” Nature Chemical Biology 13 (8): 858-66.
6. Bloom, Jesse D., Sy T. Labthavikul, Christopher R. Otey, and Frances H. Arnold. 2006. “Protein Stability Promotes Evolvability.” Proceedings of the National Academy of Sciences of the United States of America 103 (15): 5869-74.
7. Bloom, Jesse D., Jonathan J. Silberg, Claus O. Wilke, D. Allan Drummond, Christoph Adami, and Frances H. Arnold. 2005. “Thermodynamic Prediction of Protein Neutrality.” Proceedings of the National Academy of Sciences of the United States of America 102 (3): 606-11.
8. Crameri, A., S. A. Raillard, E. Bermudez, and W. P. Stemmer. 1998. “DNA Shuffling of a Family of Genes from Diverse Species Accelerates Directed Evolution.” Nature 391 (6664): 288-91.
9. Dawson, Natalie L., Tony E. Lewis, Sayoni Das, Jonathan G. Lees, David Lee, Paul Ashford, Christine A. Orengo, and Ian Sillitoe. 2017. “CATH: An Expanded Resource to Predict Protein Function through Structure and Sequence.” Nucleic Acids Research 45 (D1): D289-95.
10. Drummond, D. Allan, Jonathan J. Silberg, Michelle M. Meyer, Claus O. Wilke, and Frances H. Arnold. 2005. “On the Conservative Nature of Intragenic Recombination.” Proceedings of the National Academy of Sciences of the United States of America 102 (15): 5380-85.
11. Goodfellow, Ian J., Jean Pouget-Abadie, Mehdi Mirza, Bing Xu, David Warde-Farley, Sherjil Ozair, Aaron Courville, and Yoshua Bengio. 2014. “Generative Adversarial Networks.” http://arxiv.org/abs/1406.2661.
12. Graves, Alex. 2013. “Generating Sequences With Recurrent Neural Networks.” http://arxiv.org/abs/1308.0850.
13. Gulrajani, lshaan, Faruk Ahmed, Martin Arjovsky, Vincent Dumoulin, and Aaron Courville. 2017. “Improved Training of Wasserstein GANs.” http://arxiv.org/abs/1704.00028.
14. Guo, H. H., J. Choe, and L. A. Loeb. 2004a. “Protein Tolerance to Random Amino Acid Change.” Proceedings of the National Academy of Sciences. https://doi.org/10.1073/pnas.0403255101.
15. 2004b. “Protein Tolerance to Random Amino Acid Change.” Proceedings of the National Academy of Sciences. https://doi.org/10.1073/pnas.0403255101.
16. Hansson, Lars O., Robyn Bolton-Grob, Tahereh Massoud, and Bengt Mannervik. 1999. “Evolution of Differential Substrate Specificities in Mu Class Glutathione Transferases Probed by DNA Shuffling 1 1 Edited by R. Huber.” Journal of Molecular Biology. https://doi.org/10.1006/jmbi.1999.2607.
17. He, Kaiming, Xiangyu Zhang, Shaoqing Ren, and Jian Sun. 2015. “Deep Residual Learning for Image Recognition.” http://arxiv.org/abs/1512.03385.
18. Huang, Hua, Chetanya Pandya, Chunliang Liu, Nawar F. Al-Obaidi, Min Wang, Li Zheng, Sarah Toews Keating, et al. 2015. “Panoramic View of a Superfamily of Phosphatases through Substrate Profiling.” Proceedings of the National Academy of Sciences of the United States of America 112 (16): E 1974-83.
19. Ioffe, Sergey, and Christian Szegedy. 2015. “Batch Normalization: Accelerating Deep Network Training by Reducing Internal Covariate Shift.” http://arxiv.org/abs/1502.03167.
20. Jang, Eric, Shixiang Gu, and Ben Poole. 2016. “Categorical Reparameterization with Gumbel-Softmax.” http://arxiv.org/abs/1611.01144.
21. Jolicoeur-Martineau, Alexia. 2018. “GANs beyond Divergence Minimization.” http://arxiv.org/abs/1809.02145.
22. Karras, Tero, Timo Aila, Samuli Laine, and Jaakko Lehtinen. 2017. “Progressive Growing of GANs for Improved Quality, Stability, and Variation.” http://arxiv.org/abs/1710.10196.
23. Keefe, A. D., and J. W. Szostak. 2001. “Functional Proteins from a Random-Sequence Library.” Nature 410 (6829): 715-18.
24. Kiefer, J., and J. Wolfowitz. 1952. “Stochastic Estimation of the Maximum of a Regression Function.” Annals of Mathematical Statistics 23 (3): 462-66.
25. Kingma, Diederik P., and Jimmy Ba. 2014. “Adam: A Method for Stochastic Optimization.” http://arxiv.org/abs/1412.6980.
26. Lim, Jae Hyun, and Jong Chul Ye. 2017. “Geometric GAN.” http://arxiv.org/abs/1705.02894.
27. Maas, Andrew L. 2013. “Rectifier Nonlinearities Improve Neural Network Acoustic Models.” https://pdfs.semanticscholar.org/367f/2c63a6f6a10b3b64b8729d601e69337ee3cc.pdf.
28. Maaten, Laurens van der, and Geoffrey Hinton. 2008. “Visualizing Data Using T-SNE.” Journal of Machine Learning Research: JMLR 9 (Nov): 2579-2605.
29. McCloskey, Douglas, and Baljit K. Ubhi. 2014. “Quantitative and Qualitative Metabolomics for the Investigation of Intracellular Metabolism.” SCIEX Tech Note, 1-11.
30. Mescheder, Lars, Andreas Geiger, and Sebastian Nowozin. 2018. “Which Training Methods for GANs Do Actually Converge?” http://arxiv.org/abs/1801.04406.
31. Miyato, Takeru, Toshiki Kataoka, Masanori Koyama, and Yuichi Yoshida. 2018. “Spectral Normalization for Generative Adversarial Networks.” http://arxiv.org/abs/1802.05957.
32. Nair, Vinod, and Geoffrey E. Hinton. 2010. “Rectified Linear Units Improve Restricted Boltzmann Machines.” In Proceedings of the 27th International Conference on International Conference on Machine Learning, 807-14. Omnipress.
33. Rennell, D., S. E. Bouvier, L. W. Hardy, and A. R. Poteete. 1991. “Systematic Mutation of Bacteriophage T4 Lysozyme.” Journal of Molecular Biology 222 (1): 67-88.
34. Riesselman, Adam J., John B. Ingraham, and Debora S. Marks. 2018. “Deep Generative Models of Genetic Variation Capture the Effects of Mutations.” Nature Methods 15 (10): 816-22.
35. Rockah-Shmuel, Liat, Ágnes Tóth-Petróczy, and Dan S. Tawfik. 2015. “Systematic Mapping of Protein Mutational Space by Prolonged Drift Reveals the Deleterious Effects of Seemingly Neutral Mutations.” PLoS Computational Biology 11 (8): e1004421.
36. Romero, Philip A., and Frances H. Arnold. 2009a. “Exploring Protein Fitness Landscapes by Directed Evolution.” Nature Reviews. Molecular Cell Biology 10 (12): 866-76.
37. 2009b. “Exploring Protein Fitness Landscapes by Directed Evolution.” Nature Reviews. Molecular Cell Biology 10 (12): 866-76.
38. Romero, Philip A., Andreas Krause, and Frances H. Arnold. 2013. “Navigating the Protein Fitness Landscape with Gaussian Processes.” Proceedings of the National Academy of Sciences of the United States of America 110 (3): E193-201.
39. Sarkisyan, Karen S., Dmitry A. Bolotin, Margarita V. Meer, Dinara R. Usmanova, Alexander S. Mishin, George V. Sharonov, Dmitry N. lvankov, et al. 2016. “Local Fitness Landscape of the Green Fluorescent Protein.” Nature 533 (7603): 397-401.
40. Shafikhani, S., R. A. Siegel, E. Ferrari, and V. Schellenberger. 1997. “Generation of Large Libraries of Random Mutants in Bacillus Subtilis by PCR-Based Plasmid Multimerization.” Bio Techniques 23 (2): 304-10.
41. Shi, Wenzhe, Jose Caballero, Ferenc Huszár, Johannes Totz, Andrew P. Aitken, Rob Bishop, Daniel Rueckert, and Zehan Wang. 2016. “Real-Time Single Image and Video Super-Resolution Using an Efficient Sub-Pixel Convolutional Neural Network.” http://arxiv.org/abs/1609.05158.
42. Steinegger, Martin, and Johannes Soding. 2017. “MMseqs2 Enables Sensitive Protein Sequence Searching for the Analysis of Massive Data Sets.” Nature Biotechnology 35 (11): 1026-28.
43. Taverna, Darin M., and Richard A. Goldstein. 2002. “Why Are Proteins Marginally Stable?” Proteins 46 (1): 105-9.
44. Tran, Dustin, Rajesh Ranganath, and David M. Blei. 2017. “Hierarchical Implicit Models and Likelihood-Free Variational Inference.” http://arxiv.org/abs/1702.08896.
45. Ulyanov, Dmitry, Andrea Vedaldi, and Victor Lempitsky. 2016. “Instance Normalization: The Missing Ingredient for Fast Stylization.” http://arxiv.org/abs/1607.08022.
46. Vaswani, Ashish, Noam Shazeer, Niki Parmar, Jakob Uszkoreit, Llion Jones, Aidan N. Gomez, Lukasz Kaiser, and Illia Polosukhin. 2017. “Attention Is All You Need.” http://arxiv.org/abs/1706.03762.
47. Voigt, Christopher A., Carlos Martinez, Zhen-Gang Wang, Stephen L. Mayo, and Frances H. Arnold. 2002. “Protein Building Blocks Preserved by Recombination.” Nature Structural Biology 9 (7): 553-58.
48. Williams, Ronald J. 1992. “Simple Statistical Gradient-Following Algorithms for Connectionist Reinforcement Learning.” Machine Learning 8 (3-4): 229-56.
49. Yang, Kevin K., Zachary Wu, and Frances H. Arnold. 2019. “Machine-Learning-Guided Directed Evolution for Protein Engineering.” Nature Methods 16 (8): 687-94.
50. Zhang, Han, Ian Goodfellow, Dimitris Metaxas, and Augustus Odena. 2018. “Self-Attention Generative Adversarial Networks.” http://arxiv.org/abs/1805.08318.
51. WO2019097014

1. A method for production of functional synthetic protein sequences, comprising the steps of:
a) defining the approximate sequence space boundaries for the synthetic sequences to be produced by selecting a plurality of existing protein sequences,
b) processing the selected protein sequences,
c) approximating the unknown true distribution of amino acids of the pre-processed sequences using generative adversarial networks,
d) obtaining synthetic protein sequences from the approximated distribution,
e) processing of the obtained protein sequences.

2. A method according to claim 1, wherein the produced functional synthetic protein sequences are enzymes.

3. A method according to claim 1, wherein the pre-processing of the selected protein sequences includes filtering of sequences by their biological characteristics.

4. A method according to claim 1, wherein self-attention layers are included in the generative adversarial network architecture.

5. A method according to claim 1, wherein dilated convolutional layer are included in the generative adversarial network architecture.

6. A method according to claim 1, wherein generative adversarial network layers are normalized using spectral normalization.

7. A method according to claim 1, wherein during the generative adversarial network training the under-represented training sequence clusters are dynamically up-sampled.

8. A method according to claim 1, wherein additional information is provided to the discriminator and generator networks.

9. A method according to claim 1, wherein the amino acids are encoded using one-hot encoding.

10. A method according to claim 9, wherein the generator network produces one-hot encoded outputs using differentiable discrete approximation.

11. A method according to claim 1, wherein the amino acids are encoded using embeddings.

12. A method according to claim 1, wherein in the processing of the obtained synthetic protein sequences includes filtering the sequences by the score assigned by the discriminator network.

13. A method according to claim 1, wherein in the processing of the obtained synthetic protein sequences includes filtering the sequences by subjecting them to machine learning models.

14. Use of the functional protein sequences produced by the method described in claim 1 for experimental protein screening.

15. Use of the functional protein sequences produced by the method described in claim 1 for data augmentation.


SEQUENCE LISTING (18 sequences)

SEQ ID NO: 1 - protein, 325 aa, Candidatus Protochlamydia naegleriophila
MIAPKKVVITGASGRIGYQLVFLIAQGLLLGADQPIILCMLEKESNQQKCRGIQMELEDSIFPLLRAVHYTHDLDCAFTGADYVFFCGAKTCHDPKTRALVQAENWEALTLQGETINRVCSHQTLFLMVANPCNTNTLALIKAAPNIPPMNFHSMMRLDLNRARQFIAEKAQCLSQDVEHMLIWGNHSPRVVPDWSHVKIMASPISLLVDNNWLSSHFIHAVQKRGMAITQNMGLSSCASAAYAAIEAMKALIVPTAQNSFFCTGMYSSSNPFEFDQDLVFGLPCRTITRGVYEICEDYSPHPLLKELIKCSEAELLEERARLKK

SEQ ID NO: 2 - protein, 315 aa, Aliivibrio fischeri
MKVSYYEVKERLIRKFIASGLAWDDANWVTDVLISSEQRGDKSHGIKHAKNIFDVINSECYIAQAPIIHDERSITILDGQNSIGPIVAKQAIDIAIKKAKKYGTAAISLRSSNHLFSLSHYVRYIANNNMIGFICSSSSPAMAAPNSLNATIGTNPFAFGAPSSKDPIVIDMSSTNVARGKIKEYKDAELDIPVSWALDEYGNPTTCAIEALKGTLSPLGGYKGFALGCMIDIFSSVLSGSAFSTQITGTSLHMEEADVNKKGDFLFVLDISKFIQLSEFKIRMDEFIHIIESNGGYIPGTNYINNQFADIEILN

SEQ ID NO: 3 - protein, 314 aa, Rhodococcus sp.
MAEGVESGGAPARAVVVGAGHVGMISAMRLADADVFDEVVLVDVEPGLAAGIALDLTHTGALTGSGTRVRGAVSLEEAGPADYVIITAGKARRPGMSRSDLTAANARIVGDVAGRAAAVSPDVVLVVVTNPLDEMTYQAWRSSGLPAEQVLGMAGLLDAGRFRALAAADGAGAARELGAVALGSHGEEMVIPLSQAPEIAARLDAARLDALVDRTRTSGAEVLGLLKSGSAFFGPGTAAAHMVLAMVADSGTVVSATVRPNGEYGIGDVFVGLPVVLGRRGVRRIVELDLTPEELELLREAAKRIGERIAGLPA

SEQ ID NO: 4 - protein, 366 aa, Cedecea davisae
MASFLPASKCSSNAVDDCGISQSGSGKIVIISSLDYGRHRMELRHDFANHNKEFRMKVAVLGAAGGIGQALALLLKTQLPSGSELSLYDIAPVTPGVAVDLSHIPTDVKIKGFSGEDATPALVGADVVLISAGVARKPGMDRSDLFNVNAGIVKNLIEQVAKTAPKACIGIITNPVNTTVAIAAEVLKKAGVYDKNKLFGVTTLDIIRSNTFVAELKGKKPTDIEVPVIGGHSGVTILPLLSQIPGVSFTEQEVADLTKRIQNAGTEVVEAKAGGGSATLSMGQAAARFGLSLVRALQGESNVVECAYVEGDGEHARFFSQPLLLGKNGIVERKAIGTLSAFEKQAMEGMLDTLKKDIQLGEDFVK

SEQ ID NO: 5 - protein, 364 aa, Bifidobacterium ruminantium
MTFDCPIPRLRTFLEHLFHNHYGFTVNQAANISDALISADSHGIQSHGVQRLAMYDAKLKAGTIIAGHEGRIISQSDATSLIDGELGMGQVIAHKAMGLSIETARRYGIGIVAVRNSNHFGTAGHWAHMALEEGFIGLVSTNSNPLTVPTFGVCPFGGSNPIAFSAPGDPEDFEFDAATSTVSLGKIEVLRKTGNAIPGQWAVDEQGNIDISADHVMRNITRPERIGGLTPIGGPGEVNGGYKGYGLGLVIEILTGLLAGGPLSADLKGKAISHTFLTIDPARFGDSRMFKFAVSDMQRRLRSLPSEPGKQILVPGDKERVALRQTQQHGVPIDEVTAHQIAQIADEAGLPVPWKDTTAKKTAS

SEQ ID NO: 6 - protein, 351 aa, Spirochaeta sp.
MTVDNSISRNIFSNHSVPLCHCRILARRSAEPGNCKTPCREVLVGSGKIFIVGAGHVGATAAAVMAERKLGDIHLYDITRDRAVGNAMDINQSSPFYGTDSLVRGSNDIEDMHDSDVVVVAAGVPRHNGMTRLDLLKKNIDVIDGVGRNIQKFCPHAQVFIVTNPVDVLTWYAGTRWPDLHIFGLGCTLDTIRFQFFLAEALKVSVDSVSSIVIGTHDEHMIPLIEYATVCGLPVQTLLDESAAAEIMRNTIEAGSDIVRKLKERSGFYAAAAAITQVVESIVLNKRHIMPLSIRCNGEYGHRDICLALPVIVGREKNKEIVEVHLSPEIRMKLDNCAQSMAAVLQNIRIA

SEQ ID NO: 7 - protein, 352 aa, Delftia sp.
MTDSLNPLTDAVRMPAAALQAWAAGVLQAFDVSACHAQATAQALVRTSLRGVDTHGVARLPPYVQRIREGTIHTRAKPSLQERNGLLRCHGDHALGQPVLELALQACLDRAASQAVVACHIEACGHLGALGVLLLPAVEQGCMALLCQRTPPIMAMHGATRPALGNNPIAFAAPVAGRPPLVFDMALSAVARGAVLAAARDGHAQIPPHWALDAHGHPTTDPRAALQGAMQPMAGHKGLGLAMVVECLAGALGGGLFQPAVPGSAEGSAPNACAFLLVINPALAAPSPRGFDNYMASWITGLLAAQGTQGRYPGLRQHECELQRRAEGIPLSSALINELQALARLSGVALAL

SEQ ID NO: 8 - protein, 313 aa, Candidatus Omnitrophica
MSLPKVTILGAGQVGATTAHLLVLKGLADVVLIDVVEGLAAGKALDLLQAAAIEELAVSVTGTTDMGAMAGSRLVVITAGLARKPGMSRDDLLAANANIVGPLADQIARVAPEAVVIVVTNPLDVMTQLVLQRTGFPRQRVMGMAGMLDTGRLRAFISQRLTVSPSEVQAVVLGSHGDLMVCLTDAIRVQGAPLSQRLSSQEIDQLVARARDGGAEIVSLLKTSSAYYAPASGVVHMISAVLGGTRRVLSACVLLEGEYGLRDVCIGVPVEVGTEGIARVVEQPLSPQAREALHRAADQVRAQVKQLLTLKPR

SEQ ID NO: 9 - protein, 313 aa, Artificial Sequence
  Generated malate dehydrogenase
MSKVTVVGAGNVGATCANVLAFNQVADEVVMLDVKEGVSEGKAMDMMQTAQLLEFDTTVVGCTNDYSKTANSDVVVITSGIPRKPGMTREELIGVNAGIVKEVAENVLKYSPNAILVVISNPMDTMTYLSLKSLGLPKNRVIGMGGALDSSRFKYFLSQALGCNPKDVEGMVIGGHGDTTMIPLTRLATYNGMPVTQFLSAEQLEKVAADTMVGGATLTGLLGTSAWYAPGASAAYVVKSIVNDRKKLIPCSVYLEGEYGESDICIGVPVVIGKNGVEKIVELELNEDEKDAFAASAEAVRAMNAVLKELNVI

SEQ ID NO: 10 - protein, 308 aa, Artificial Sequence
  Generated malate dehydrogenase
MNRKVTVVGSGNVGATAARRIATKELADVVLLDILEGVPQGKGLDMLEANPVEGSDSCVLGTNDYADTANSDIVVITAGLARKPGMSRDDLLMKNAAIIKSVTEQIVKHSPNCIIVPVTNPLDAMCQVVYRVSGFPRERVVGMAGILDSARMRAFIAEELNVSVENTHAFVLGGHGDTMVPLPRYSTVAGIPITELMDSATIDQIVKRTAGGGAEIVALLGTGSAYYSPGSSIVEMVEAILKDKKKILPCAVFLQGEYGIHGLYVGVPVKLGAGGVEDIIEITLTPDERAALEKSAEAVKELVDVIKI

SEQ ID NO: 11 - protein, 308 aa, Artificial Sequence
  Generated malate dehydrogenase
MRKKVTIVGSGNVGATAAHWIASKELADVVLIDIIEGVPQGKGLDLLEAMPIEKRDSYILGTNDYADTANSDIVVITAGIPRKPGMSRDDLLNTNWGIMKDVVGKVVQVSPNCILIVVSNPLDAMAQAALKLSGFARERVIGMAGVLDSARMRAFIAWELKVSVENTTAFVLGGHGDTMVPLPRYSTVAGIPITELMDAATLARIVQRTRDGGAEIVKLLKTGSAYYAPSAAITEMVEAILKDKKKILPCAAYLQGEYGINGLFVGVPVKLGAKGIEQVIEIKLTPEEKAGLDRSAAAVKELVGVIGV

SEQ ID NO: 12 - protein, 320 aa, Artificial Sequence
  Generated malate dehydrogenase
MARQKIALIGSGQIGGTLAHLAALKELGDVVLFDIVEGIPQGKALDIAESSPVDGVDVNLTGTNDYADIAGADVCIVTAGVPRKPGMSRDDLLGINLKVMKSVGEGIKKYAPEAFVICITNPLDAMVWALQKFSGLPACKVVGMAGVLDSARFRYFLSEEFNVSVEDVTAFVLGGHGDTMVPLARYSTVAGIPLPDLVKMGCTSQDRLDKIIQRTRDGGAEIVGLLKTGSAFYAPAASAIQMAESYLKDKKRVLPVAAWLSGQYGVKDMYVGVPTVIGANGVEKVIEIDLDKNEKAQFDKSVASVAGLVEACIGIAPALK

SEQ ID NO: 13 - protein, 321 aa, Artificial Sequence
  Generated malate dehydrogenase
MARNKIALIGSGMIGGTLAHLAGLKELGDVVLFDIAEGTPQGKGLDIAESSPVDGFDAKFTGANDYAAIEGADVVIVTAGVPRKPGMSRDDLLGINLKVMEQVGAGIKKYAPEAFVICITNPLDAMVWALQKFSGLPACKVVGMAGVLDSARFRYFLSEEFNVSVKDVTAFVLGGHGDDMVPLARYSTVAGIPLPDLVKMGCTSQDRLDAIVQRTRDGGAEIVGLLKTGSAFYAPAASAIAMAESYLKDQKRVLPVAAWLSGQYGVKDMYVGVPTVIGAGGVERVVEIDLDKDEKALFAKSVASVAGLIEACIGIAPSLKK

SEQ ID NO: 14 - protein, 326 aa, Artificial Sequence
  Generated malate dehydrogenase
MGKTVKVAVTGAAGQIGYSLLFRIASGQMFGADTAVEIQMLELEAAIPAAKGVIMELEDCAFPLLQKVTVSSDLDTAFKDINWALLVGSVPRKAGMERGDLLKINGGIFVNQGKAIEKNAASDVRILVVGNPCNTNCLIAMNNAKGVPQDRWFAMTKLDENRAKSQLASKAGVPVKEVTHLGIWGNHSSTQYPDFYNAKISGKPVTDVISDWEWLKGDFIKNVQQRGAEIIKARGASSAASAANGVVDTVRGIITPTAPGDWFSAAIVSDGSYGAEKGLIFGFPLKSDGKKVEIIQGLSLNDFAKEKFKITHDELVSERNEVKEML

SEQ ID NO: 15 - protein, 327 aa, Artificial Sequence
  Generated malate dehydrogenase
MKTPIRVAVTGAAGQIGYAALFRIANGDMLGREQPVILQLLDLPQAQGMLRGVMMELEDCAFPLLQGVIITDDPKVAFRDTEIALLIGARPRTKGMERKDLLEANGAIFSAQGKLLNEYAARHVKVLVVGNPANTNALIAMKNAPDLDPRNFSSMMRLDHNRAISQVAQKLGQPVSSIKKMIVWGNHSGTQYPDLDHAEIGGKKVWNLIDDQAWIENYFIPTVQKRGAAVIEARGLSSAASAANAAICHIRDWILGAHGNDWVTMSVPSDGSYGIPEGVIYGFPVTCRNGEYKIVQGLDISEIGQKCMDENYRELEEEREMVQQLLS

SEQ ID NO: 16 - protein, 313 aa, Artificial Sequence
  Generated malate dehydrogenase
MSKVTVVGAGNVGATCANVLAFNKVADEVVMLDVKEGVSEGKAMDMMQTAQLLGFDTTVVGCTNDYEKTANSDVVVITSGIPRKPGMTREELIGVNAGIVKSVAQNVLKYSPDAILVVISNPMDTMTYLSLKSLGLPKNRIIGMGGALDSSRFKYYLSQALGHNANDIEGMVIGGHGDTTMIPLTRLATYKGMPVSNFLSAETLDKVAADTMVGGATLTGLLGTSAWYAPGAAGAYVVDSIVRDEKKLITCSVYLEGEYGQSDISIGVPVVIGKNGCEKVVEIKLNEKEKEAFSKSAEAVRATNNVLREINVI

SEQ ID NO: 17 - protein, 348 aa, Humibacter sp.
MSTPSSTPHPSASVTESTRAAHPPVTVTVTGAGGQIGYALLFRIAAGDMLGASTPVRLRLLEVPQGLASAEGAALELQDGAFPLLESLEVTDSPERAFDGTNVALLVGSRPRAAGMERADLLAANGGIFGPQGAALNAHAADDVRVLVVGNPANTNALIAQQNAPDIPADRFTALTRLDHNRALAQLAAKLEVPVSSVRDVTIWGNHSTTQYPDVFHALAEERPVRDLVDEAWLAEEFIPRVAGRGAEIIDARGSSSAASAASAAIDHVRDWVHGTRNGWTSVALPSTGAYGVPEGIVSSFPVHSIDGRWVVVEGLDVNAFSRRRIDLSVDELLEERAAVAARGLLPS

SEQ ID NO: 18 - protein, 331 aa, Gamma proteobacterium
MRREPVKAPVRVAVTGAAGQISYSLLFRIAAGEMLGPDQPVILQMLEITPALEALNGVAMELDDCAFPLLAGMVQTDDPNVAFKDADYALLVGARPRGPGMERKDLLEANAAIFSVQGKAINDNASRNINVLVVGNPANTNALIAQRNAPDLDPRQFTAMTRLDHNRALSQLAQKTGTTVNDVTRMIIWGNHSSTQYPDIHHSSVKGQAGIDLVDQDWYETTFIPDVQQRGAAIIKARGASSAASAANAAIGHMRSWALGTAEGDWVSMGIYSDGSYGIAKGLIYSFPCVCKNGDWEIVQGLAINDFSQARMKASEDELLEERDGVSHLLP